(19) United States
(12) Patent Application Publication
(10) Pub. No.: US 2025/0099516 A1
(43) Pub. Date: Mar. 27, 2025

(54) AGENT OF ANTI-INFECTIVE TREATMENT OF HUMANS OR ANIMALS, ITS MANUFACTURING METHOD AND USE

(71) Applicant: AVSYSTEMS INC., Stamford, CT (US)

(72) Inventors: Albina Aleksandrovna KORNILOVA, Moscow (RU); Igor Vadimovich KORNILOV, Moscow (RU)

(21) Appl. No.: 18/723,722

(22) PCT Filed: Dec. 28, 2021
(86) PCT No.: PCT/IB2021/062381

§ 371 Date: Jun. 24, 2024

(51) Int. Cl. A61K 35/747; A61K 9/00; A61K 35/745; A61P 31/16; C12N 1/20; C12N 13/00

(52) U.S. Cl. CPC A61K 35/747; A61K 9/0014; A61K 9/0053; A61K 35/745; A61P 31/16; C12N 1/20; C12N 13/00

(57) ABSTRACT

An agent of anti-infective treatment of humans or animals, which has a suppressive action to the infection, while activating natural human microflora and local immune response, includes metabolites isolated by symbiotic microorganisms of human microbiota or microorganisms friendly to human organism because of previously realized stress (life-threatening) effect on these organisms. Strains of microorganisms approved for use included in the human microbiome or friendly to normal human microflora are grown in a nutrient medium, placed under life-threatening physical coercion and incubated to produce protective metabolites, which are then released by separating the liquid sediment. The treatment is inserted into the body by applying on skin, and/or in the fauces, and/or epipharynx, and/or external genital organs, and/or by injecting into internal genital organs, and/or by injecting into the anus, and/or by microdrop irrigation of the lungs and/or injections, and/or placing the body in cold fog.

### BACKGROUND OF THE INVENTION

### Field of the Invention

The group of inventions relates to the field of hygiene and sanitation, more specifically to the field of public health provision, namely, to preventive interventions for the spread and treatment of bacterial and viral infections (anti-infective agents), including the coronavirus infection COVID-19 caused by SARS-CoV-2 and its variants, and to methods for making and applying a method of applying the agent of anti-infective treatment of humans or animals and stimulating their resistance to infection and other adverse influences.

### Background of the Related Art

Various disinfectants are used to prevent the spread of bacterial and viral infections.

A technical solution is known according to PRC utility model patent No. CN204275091 (published on Apr. 22, 2015), in which ultraviolet light is used as a disinfectant.

The disadvantage of the solution is the obligatory protection of eyes and exposed human skin to avoid damage by ultraviolet radiation. The unacceptability of the use of ultraviolet light to prevent COVID-19 coronavirus infection caused by the SARS-CoV-2 virus is highlighted by the World Health Organization (hereinafter to be referred to as the WHO) on the website ( . . . www . . . who . . . int/ru/emergencies/diseases/novel-coronavirus-2019/advice-for-public).

There is a known technical solution according to the patent for a utility model of the Russian Federation No. 201671 (published on Dec. 28, 2020) in which ozone and a bactericidal irradiator are used as a disinfectant. The disinfection is provided by a combination of air-borne ozone mixture and diffused ultraviolet radiation.

A disadvantage of this technical solution is the fact that the used ozone is a gas, toxic when inhaling, which refers to substances of the first class hazard with a sharp-directed mechanism of action, and maximum residue limit (MRL) of ozone in the air is 0.1 mg/m3 (GOST 12.1.005-88). From this perspective when using the device it is necessary to apply individual means of eye and respiratory system protection.

Disinfectants of chloroxygen and hydropyroxide compounds with pH 7.0-8.0 and concentration of active substances not more than 0.05% sprayed on human clothing and skin in the form of cold fog in a disinfection booth are known from the prior art http: . . . a-zdor . . . ru/catalog/disinfectant_complexes/(published on Aug. 3, 2020).

The disadvantage of this technical solution is the fact that chloroxygen and hydropyroxide compounds in contact with skin and mucous membranes cause their irritation and can lead to dermatitis and necrosis. This excludes the use of technical solution without the use of personal eye and respiratory protection. The WHO has specifically stated on its website (https . . . www . . . who . . . int/ru/emergencies/diseases/novel-coronavirus-2019/advice-for-public) that skin application and inhalation of such disinfectants in the prevention of COVID-19 coronavirus infection caused by SARS-CoV-2 is not acceptable.

To prevent the spread of bacterial and viral infections, including the SARS-CoV-2-caused coronavirus COVID-19 infection, WHO recommends frequent hand washing with alcohol-containing agents or soap and water, indicating that this measure will eliminate possible microbial contamination of hands, including viral contamination (https . . . www . . . who . . . int/ru/emergencies/diseases/novel-coronavirus-2019/advice-for-public).

The disadvantage of all these disinfectants is their negative impact on human microbiota (hereinafter to be referred to as the microflora) (communities of symbiotic microorganisms (hereinafter to be referred to as the symbionts)) living in and on the human body, including the skin), which is a part of the human immune system. All these disinfectants suppress not only infectious but also beneficial microflora, reducing a person's immunity in general.

It is not known from the prior art of methods of target generation of metabolites of symbiotic microorganisms that enhance the activity of symbiotic microorganisms on or in the human body.

Methods for insertion of metabolites of symbiotic microorganisms into the digestive system to restore the microbiota of the gastrointestinal tract are known from the prior art.

The disadvantage of this technical solution is that the digestive system destroys some of the useful metabolites.

### SUMMARY OF THE INVENTION

The technical result achieved by the group of inventions is the creation of the agent of anti-infective treatment of humans or animals, which has a suppressive action to the infection, while activating natural human microflora and local immune response, as well as methods of its manufacture and use.

The technical result is achieved by a solution of anti-infective treatment of humans or animals, which consists of metabolites excreted by symbiotic microorganisms of human microbiota or microorganisms friendly to human organism as a result of previously implemented stress (life-threatening) effect on these organisms.

Such metabolites, including those containing specific protective substances having the structure of peptidoglycans, glycoproteins and peptides, have a stimulating effect on the immune response of human cells by binding to the surface proteins of these cells. At the same time, they are modulators of the growth of most representatives of normal human microflora as molecules providing quorum sensing (Patent of the Russian Federation No. 2534617, published on Nov. 27, 2014). The resulting metabolome (a complex of metabolites) also contains cytostatic and anti-inflammatory substances. Besides, the mentioned substances in mixture with aminoacids, triglycerides, metalloproteins and proteins included into exo- and endo-metabolites cause suppressive action on pathogenic and conditionally pathogenic microorganisms and viruses.

The technical result is also achieved by the manufacturing method the agent of anti-infective treatment for humans or animals, where the strains of microorganisms approved for use included in the human microbiome or friendly to normal human microflora are grown in a nutrient medium, placed under life-threatening physical conditions, incubated to produce protective metabolites, which are then excreted by separating the liquid sediment.

Preferably, as a nutrient medium, a composition consisting of the following components with a biogenic concentration from 0.001 to 30 g/l is used:
Pancreatic casein hydrolysate (20.0-30.0 g/l);
Baker's yeast extract (2.5-5.0 g/l);
Bactopeptone (8.0-12.0 g/l);
Meat extract (8.0-12.0 g/l);
Fish hydrolysate (5.0-7.0 g/l);
Tween 80 (0.5-1.0 g/l);
Yeast extract (5.0-10.0 g/l);
Yeast autolysate (25.0-50.0 g/l);
Multivitamin complex (1.0-1.5 g/l);
Food grade polyols (1-10 g/l);
Vegetable oil (0.05-0.15 g/l);
Flavonoids (0.1-0.2 g/l);
Glucose (7.5-20 g/l);
Lactose (0.5-2.5 g/l);
Cysteine (0.4-0.5 g/l);
Starch (0.4-0.5 g/l);
Pectin (0.001-0.003 g/l);
Bactotripton (0.5-1.0 g/l);
Rezasurin (0.00005-0.0001 g/L);
Na₃C₆H₅O₇ (0.02-0.06 g/l);
CaCO₃ (0.5-1.0 g/l);
Red chlorophenol (0.04-0.1 g/l);
Skim milk (50-100 g/l);
NaCl (0.01-0.1 g/l);
Ammonium citric acid (0.5-2.0 g/l);
Ammonium acetic acid (1.0-3.0 g/l);
Ascorbic acid (0.1-0.5 g/l);
Sodium acetic acid (1.0-5.0 g/l);
MgSO₄·7H₂O (0.1-0.5 g/l);
MnSO₄·5H₂O (0.03-0.05 g/l);
Na₂HPO₄ (1.0-2.0 g/l);
KH₂PO₄ (0.3-0.7 g/l);
K₂HPO₄ (0.3-0.7 g/l);
FeSO₄·7H₂O (0.035-0.075 g/l);
Agar (0.1-15.0 g/l);
Micronutrients (0.5-1.0).

Preferably, from 2 to 100 human symbiont species are mixed, in equal parts, or with a variation of up to 99.9% of the part, i.e. 1:1 to 1:0.001 parts or use biomass of a single strain.

Preferably, each strain is grown separately using a method generally accepted in biotechnology under conditions optimal for the species of microorganisms until the number of live cells is at least 1·10⁹ CFU/ml.

Preferably, as a life-threatening physical coercion, temperature shock (temperature change by 15-40° C. in a time interval from 1 to 60 minutes), and/or gravity, and/or shock effect, and/or shock and cavitation effects are applied.

Preferably, one chooses the time of life-threatening treatment of the strain biomass mixture from the range of 1-20 minutes.

Preferably, the mixture is incubated for at least 20 minutes after a life-threatening treatment at +20 to +28° C. for metabolite production prior to metabolite generation.

Preferably, the metabolites are excreted on several stages of centrifugal separation, wherein the centrifugal acceleration value is varied from 3000 G to 21000 G, the centrifugal time is from 1 min to 60 min and the temperature is from +2 to +25° C.

Preferably, the liquids obtained after separation from the sediment are combined in proportions according to the material balance formula when liquids of different densities are mixed:

$${V}_{k} \cdot {p}_{k} = {V}_{1} \cdot {p}_{1} + {V}_{2} \cdot {p}_{2} + {V}_{3} \cdot {p}_{3} + {V}_{n} \cdot {p}_{n} ,$$

where, V₁ and Vₙ are volume of supernatant liquid No. 1 and n, ml; p₁ and pₙ are density of supernatant liquid No. 1 and n, g/ml; V_{κ} is the final volume, ml; pₖ is the final density, g/ml.

Technical result is also achieved by a method of using the agent of anti-infective treatment for humans or animals, where the developed agent of anti-infective treatment for humans or animals is inserted into the body by applying on skin, and/or in the fauces, and/or epipharynx, and/or external genital organs, and/or by injecting into internal genital organs, and/or by injecting into the anus, and/or by microdrop irrigation of the lungs and/or injections, and/or placing the body in a cold fog.

In one of the usage variants the agent of anti-infective treatment for humans or animals is pre-mixed with a matrix of additive, while the agent of anti-infective treatment for humans or animals is mixed with a matrix of additive under aseptic conditions until a biologically active concentration with a metabolite concentrate content in the matrix of between 0.1 and 99.9% is achieved.

Preferably, the agent of anti-infective treatment of humans or animals or the agent of anti-infective treatment of humans or animals mixed with the matrix is subjected to sterilizing filtration after manufacture.

In one of the implementation methods an isotonic solution, and/or natural mineral water, and/or an aqueous solution of sea salt is used as an additive.

The above variant of the method is used in the production of sprays and gargles for application on the skin, in the oropharynx, nasopharynx, genitals, skin around the eyes, on the inner surface of the lungs.

In one of the implementation methods lozenges, pastilles or tablets for oral resorption are formed from the concentrate mixed with the matrix of additive.

In one of the implementation methods the concentrate mixed with the matrix of additive is applied by penetration method with a possible subsequent drying stage to wipes, plasters or patches.

In one of the implementation methods for injection, the water for injection is used as an additive.

In one of the implementation methods, the concentrate mixed with the matrix of additive is applied by penetration method with a possible subsequent drying stage to nasal or vaginal tampons.

In one of the implementation methods suppositories including those for anal or vaginal insertion are formed from the concentrate mixed with the matrix of additive.

Additional features and advantages of the invention will be set forth in the description that follows, and in part will be apparent from the description, or may be learned by practice of the invention. The advantages of the invention will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### DETAILED DESCRIPTION OF PREFERRED EMBODIMENTS

Reference will now be made in detail to the preferred embodiments of the present invention.

The group of inventions is implemented in the agent of anti-infective treatment of humans or animals obtained as a result of a short-term life-threatening (shock and cavitation wave) impact on a mixture of biomasses of bacteria included in the human microbiome or friendly to normal human microflora.

The biomass of probiotic bacteria was used as a source of protective metabolites, Bifidobacterium bifidum, Lactobacillus delbrueckii subsp. Bulgaricus, Lactobacillus acidophilus with live cells not less than 1.0×10⁹ CFU/ml and not more than 9.9×10⁹ CFU/ml, grown on separate nutrient media, which includes biogenic for each strain sets of substances from the following list of components with concentration from 0.001 to 30 g/l:
Pancreatic casein hydrolysate (28.0 g/l);
Baker's yeast extract (4.0 g/l);
Bactopeptone (9.0 g/l);
Meat extract (9.0 g/l);
Fish hydrolysate (6.5 g/l);
Tween 80 (0.6 g/l);
Yeast extract (6.5 g/l);
Yeast autolysate (34.0 g/l);
Multivitamin complex (1.3 g/l);
Food grade polyols (9.5 g/l);
Vegetable oil (0.15 g/l);
Flavonoids (0.1 g/l);
Glucose (20 g/l);
Lactose (2.5 g/l);
Cysteine (0.45 g/l);
Starch (0.45 g/l);
Pectin (0.003 g/l);
Bactotripton (0.75 g/l);
Rezasurin (0.0001 g/L);
Na₃C₆H₅O₇ (0.03 g/l);
CaCO₃ (0.5 g/l);
Red chlorophenol (0.05 g/l);
Skim milk (75 g/l);
NaCl (0.1 g/l);
Ammonium citric acid (2.0 g/l);
Ammonium acetic acid (2.5 g/l);
Ascorbic acid (0.5 g/l);
Sodium acetic acid (1.5 g/l);
MgSO₄·7H₂O (0.3 g/l);
MnSO₄·5H₂O (0.04 g/l);
Na₂HPO₄ (1.0 g/l);
KH₂PO₄ (0.6 g/l);
K₂HPO₄ (0.6 g/l);
FeSO₄·7H₂O (0.04 g/l);
Agar (14.0 g/l);
Micronutrients (0.9 g/l).

The biomasses of microorganisms were mixed in equal parts. The total biomass mixture was subjected to a life-threatening impact using a shock and cavitation equipment producing low-frequency and high-frequency vibrations with frequencies in the range of 8 . . . 20 kHz and 70 . . . 250 MHz, respectively.

Distilled water (electrical resistivity 2 kOhm·m), a volume of from 50 to 250 liters, was used to form the shock and cavitation impact in equipment.

The water pressure in the cavitation equipment was 250-600 atm. In addition, the shock and cavitation wave was formed as a result of supersonic water jet impact on the molybdenum plate with the initial diameter of the water jet at the channel outlet in the range from 0.3 to 1 mm.

The biomass mixture for treatment was in a plastic container. The treated surface was a circle with a diameter of 3-6 cm with a continuous change of the treated surface. This was obtained by stirring the biomass mixture continuously (80-100 rpm) with a magnetic stirrer with a sterilizable magnetic anchor during shock and cavitation wave treatment. At the same time, the distance of the biomass surface from the source of shock and cavitation waves was 15-20 cm. The time of life-threatening treatment by waves generated by shock and cavitation effect on the molybdenum plate, biomass mixture of 0.75-1.5 liters was 3 minutes.

After the stress effect, the biomass mixture was incubated for 60 min at +25° C. to produce target substances. A specific type of protective substances having the structure of peptidoglycans, glycoproteins and peptides was formed in the bacterial cells, as well as in the mixture of biomasses. The formation of protective stress proteins in response to stress and the protective role of stress proteins is confirmed by the facts of cell death in vivo and in vitro when protein synthesis inhibitors are injected during the stressor action. (Molecular Biology: stress response of the cell: textbook for higher education institutions, E. N. Proshkina, I. N. Yuraneva, A. A. Moskalev. M.: Urite Publishers, 2018, p. 101).

Then the biomass mixture was placed in storage at +2 to +8° C. until the stage when target metabolites were excreted.

The concentrate of metabolites are excreted on several stages of centrifugal separation. In addition, the centrifugal acceleration value varied from 3000 G to 21000 G, the centrifugal impact time varied from 1 min to 60 min and the temperature varied from +2 to +25° C.

The resulting concentrate of protective metabolites was mixed with isotonic solution (NaCl 9 g/l) until the content of metabolites<30% and isotonic solution>70% were obtained. The resulting mixture was subjected to sterilizing filtration with a 0.22 μm pore size membrane.

According to the results of tests of the concentrate of protective metabolites on the content of toxic elements showed that the content of mercury in the obtained concentrate of protective metabolites was less than 0.05 mg/kg, lead was less than 0.2 mg/kg, arsenic was less than 0.2 mg/kg (GOST 33022-2014).

According to the results of tests of the concentrate of protective metabolites by toxicological indicators the absence of general toxic effect (GOST 33506-2015 cl.9) is shown.

The absence of irritant and sensitizing action is shown (GOST 33483-2015).

According to the results of tests of antimicrobial activity of the concentrate of protective metabolites it is shown that the obtained concentrate of protective metabolites has antimicrobial action against Bacillus cereus, Pseudomonas aeruginosa, Staphylococcus aureus, Candida albicans. In addition, in the liquid concentrate it was reported the absence of growth of microorganisms of all test strains Mycobacterium tuberculosis, genus Trichophyton, Microsporum, Epidermophyton.

Antiviral activity of the sample against Rotavirus, Conopaviridae, Adenoviridae, Hepatitis viruses (HBV, HCV), Retroviridae, Herpesviridae, Influenzavirus (including A/H1N1, A/H5N1) was reported.

The following preclinical studies were performed with a sample of obtained concentrate of protective metabolites.

All of the studies described below were performed in four independent experiments on different days with four sample sinks of the metabolite concentrate solution and of the reference solution. In four independent experiments, complete repeatability of the results was found.

Evaluation of the cytotoxic effect of the provided metabolite concentrate solution sample and control solution in MDCK cell culture (including the presence of influenza A(H1N1)pdm09 virus) by visual inspection and using the MTT assay.

The control solution slightly changed the morphology of MDCK cells

A sample of metabolite concentrate solution during the incubation period in MDCK cell culture (including the presence of influenza A(H1N1)pdm09 virus) caused a change in cell morphology during visual inspection. At the same time, the character of morphological changes in the cell was close to the apoptosis effect. The cell membrane remained intact, as cells were not painted when being painted by trypan blue staining, but the MTT assay revealed the absence of MDCK biosynthesis in cells (including the presence of influenza A(H1N1)pdm09 virus).

It was also noted in experiments that the sample of metabolite concentrate solution completely suppressed the proliferative activity of MDCK cells during daily incubation of the sample of metabolite concentrate solution in the cell monolayer, unlike the control solution. This specimen has cytostatic properties.

Assessment of the activity of pandemic influenza A(H1N1)pdm09 virus of the 2009-2010 epidemic season by incubating mixtures of different doses of the virus 10000 TCID₅₀, 1000 TCID₅₀, 100 TCID₅₀, 10 TCID₅₀ with a metabolite concentrate solution sample and a reference solution in MDCK cell culture.

When different doses of influenza A(H1N1)pdm09 virus and a sample of metabolites concentrate/reference solution were simultaneously applied to MDCK cells, no cytopathic effect characteristic of influenza A(H1N1)pdm09 virus was detected after both 24 and 48 hours of incubation in the presence of metabolites concentrate solution. There was no dose-dependent effect of the metabolite concentrate solution sample when incubated with doses of A(H1N1)pdm09 virus 10000 TCID₅₀, 1000 TCID₅₀, 100 TCID₅₀, and 10 TCID₅₀. The activity of influenza A(H1N1)pdm09 virus in the reference solution corresponded to the virus control.

An enzyme-linked immunosorbent assay (ELISA test) with specific monoclonal antibodies to influenza A(H1N1) virus was used to confirm the observed effect. No binding of influenza A(H1N1)pdm09 virus at doses of 1000 TCID50, 100 TCID50, and 10 TCID50 with monoclonal antibodies to influenza A(H1N1) virus in the presence of a metabolite concentrate solution sample was detected.

In addition, an MTT assay was performed by incubating a mixture of the metabolite concentrate solution sample and different doses of influenza A(H1N1)pdm09 virus in comparison with the reference solution, virus and cell controls. This test showed absence/reduction of biosynthesis in MDCK cells where the preparation of metabolite concentrate specimen was present.

Thus, we observed the absence of replication of influenza A(H1N1)pdm09 virus at doses of 1000 TCID₅₀, 100 TCID₅₀, and 10 TCID₅₀ with the addition of a metabolite concentrate solution sample, which is explained by morphological changes in this cell type under the action of the metabolite concentrate solution sample.

This study of interaction of metabolite concentrate sample solution and reference solution with human erythrocytes of blood group 0(I) and in presence of influenza virus A(H1N1)pdm09 in hemagglutination reaction (HGR).

The studied sample of metabolite concentrate solution, unlike the reference solution, lysed human erythrocytes of blood group 0(I) during mixture 1:1 (no lysis of erythrocytes was observed in the reference solution).

The studied solution of metabolite concentrate solution without dilution inhibited the binding of influenza A(H1N1)pdm09 virus in HGR. There was no dose-dependent effect of the metabolite concentrate solution sample in HGR.

It is supposed that the active substances included in composition of the specimen expose the erythrocyte membrane in a certain way, resulting in lysis of erythrocyte. The detected effect of virus inhibition in HGR with initial dilution of the specimen is related to the effect of metabolite concentrate solution sample on erythrocyte membrane.

Thus, the studied sample of metabolite concentrate solution changes the morphology of MDCK cells by inhibiting cell biosynthesis. In addition, the ability of the metabolite concentrate solution sample to lyse human erythrocytes of blood group 0(I) was revealed. It is supposed that the active substances included in the composition of specimen expose the erythrocyte membrane in a certain way. As a result the erythrocyte is unable to interact with the influenza virus A(H1N1)pdm09 in HGR (inhibition of the initial solution of the metabolite concentrate sample was observed).

When evaluating the activity of pandemic influenza A virus in MDCK cell culture, the metabolite concentrate solution sample submitted for examination revealed the absence of virus replication at doses of 1000 TCID₅₀, 100 TCID₅₀, and 10 TCID₅₀. The results obtained are probably related to morphological changes in MDCK cells (including in the presence of influenza A(H1N1)pdm09 virus) under the action of the metabolite concentrate solution sample.

The metabolite concentrate solution sample inhibited the proliferative activity of MDCK cells, indicating the cytostatic properties of the specimen.

The following clinical studies were performed with a sample of concentrate solution of metabolites.

Conclusion on the results of a clinical study of a hygienic preventative spray based on concentrate of metabolites and isotonic solution in Branch No. 5 of the Federal State Budgetary Institution The Main Military Clinical Hospital named after N.N. Burdenko of the Ministry of Defense of Russia

Purpose of the study: to evaluate the effectiveness of the specimen in relation to prevention of infectious diseases among the personnel of the branch No. 5.

Number of participants: 20 persons (15 women and 5 men). All participants worked in a strict anti-epidemic zone (“red zone”) because from Jun. 15, 2021 to the present, Branch No. 5 was converted to an infectious military hospital to treat patients with the new COVID-19 coronavirus infection.

Study duration: 30 days.

The specimen was used according to the recommendations for use by applying it evenly to the surface of the face and palms of the hands and mouth, by pressing a sprayer, every 3-4 hours.

During the clinical study we evaluated general state of the participants' organisms, monitored their body temperature, blood pressure, recorded the presence or absence of catarrhal manifestations of acute respiratory viral infection, symptoms of COVID-19 with flu-like conditions, and allergic reactions.

### Results

Increased body temperature—0 persons.
Catarrhal manifestations of acute respiratory viral infection—0 persons.
Symptoms of intestinal infections—0 persons.
COVID-19 symptoms—0 persons.
Flu-like condition—0 persons.
Allergic reactions—0 persons.
Side effects—No.

From 3 participants, a decrease in hyperemia of the skin areas and contact dermatitis area was recorded.

When using the spray on affected areas of the epithelium of oropharynx, an acceleration of regeneration was noted.

Participants also noted a decrease and/or disappearance of bad breath and sinus, and a decrease in nasal congestion.

As a result of a clinical study of the specimen among the personnel of branch No. 5, it has been proven its effectiveness in preventing infectious diseases, including a new coronavirus infection COVID-19, as well as a positive effect on the skin and mucosa of the fauces and epipharynx. The result is confirmed by the fact that during the study none of the participants was diagnosed with infectious diseases. The effectiveness of the specimen in reducing skin lesions and mucous membranes has been proven in the clinical study.

Conclusion on the results of the application of oral hygiene and skin care with antibacterial effect of a spray preparation based on concentrate of metabolites and isotonic solution in the Federal State Budgetary Institution The 3rd Central Military Clinical Hospital named after A.A. Vishnevsky of the Ministry of Defense of Russia

Purpose of use: evaluation of the effectiveness of oral and skin hygiene product spray on the basis of metabolite concentrate and isotonic solution in prevention of infectious diseases among hospital personnel.

Number of participants: 28 persons, including 19 women and 9 men. All participants of the approbation working in the zone of strict anti-epidemic conditions the so-called “red zone” at the branch hospital No. 1 and No. 3, converted to treat patients with COVID-19.

The duration of the approbation was 30 days.

The following assessments were made:
general condition and well-being of the participants during approbation;
presence or absence of catarrhal manifestations of influenza and acute respiratory viral infections, intestinal infections, COVID-19 symptoms, as well as allergic reactions of local and general nature.

The spray was applied evenly to the surface of the face and hands using a sprayer, and irrigation of the oral cavity was also performed. The above manipulations were performed every 3-4 hours.

During approbation, the following results were received:

No rise of body temperature, occurrence of catarrhal and flu-like symptoms, symptoms of intestinal infection, as well as new coronavirus infection were detected in any of the participants of approbation. No allergic reactions or any other side effects of the specimen were observed.

In addition, four participants of the approbation noted that after using the spray, skin irritation symptoms associated with the need to wear protective rubber gloves at all times were significantly reduced.

Two participants noted healing and epithelialization of oropharyngeal areas that had been damaged by bites and local inflammatory reactions.

Conclusions: during application of the spray based on the concentrate of metabolites and isotonic solution in branches No. 1 and No. 3 of the Federal State Budgetary Institution The 3rd Central Military Clinical Hospital named after A.A. Vishnevsky of the Ministry of Defense of Russia the results have been received, which confirm its safety and effectiveness.

Thus, it is shown that the obtained agent of anti-infective treatment of humans or animals is safe for humans and has biogenic, antiviral and antimicrobial properties. In addition, the metabolite solution exhibits cytostatic properties and also shows pronounced regenerative and anti-inflammatory properties against the human skin epithelium.

Thus, technical results are achieved in the form of creating the agent of anti-infective treatment of humans or animals, as well as methods of its manufacture and use, which according to the studies proofs has a suppressive action on bacterial and viral infections, while activating the natural human microflora.

Having thus described a preferred embodiment, it should be apparent to those skilled in the art that certain advantages of the described method and anti-infection agent have been achieved.

It should also be appreciated that various modifications, adaptations, and alternative embodiments thereof may be made within the scope and spirit of the present invention. The invention is further defined by the following claims.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. A method of manufacturing an anti-infective agent, the method comprising:
using a nutrient medium to grow a strain of microorganism that is compatible with human microbiome or with normal human microflora;
applying life-inhibitory physical stress to the strain, wherein the stress is any of temperature shock, high-G force, a shock or shock-cavitation wave;
incubating the strain to produce protective metabolites;
releasing the protective metabolites by separating, using a centrifuge, the protective metabolites in a liquid form from the strain of microorganism using a centrifuge.

21. The method of claim 20, wherein the nutrient medium includes any of:
Pancreatic casein hydrolysate (20.0-30.0 g/l);
Baker's yeast extract (2.5-5.0 g/l);
Bactopeptone (8.0-12.0 g/l);
Meat extract (8.0-12.0 g/l);
Fish hydrolysate (5.0-7.0 g/l);
Tween 80 (0.5-1.0 g/l);
Yeast extract (5.0-10.0 g/l);
Yeast autolysate (25.0-50.0 g/l);
Multivitamin complex (1.0-1.5 g/l);
Food grade polyols (1-10 g/l);
Vegetable oil (0.05-0.15 g/l);
Flavonoids (0.1-0.2 g/l);
Glucose (7.5-20 g/l);
Lactose (0.5-2.5 g/l);
Cysteine (0.4-0.5 g/l);
Starch (0.4-0.5 g/l);
Pectin (0.001-0.003 g/l);
Bactotripton (0.5-1.0 g/l);
Rezasurin (0.00005-0.0001 g/L);
Na₃C₆H₅O₇ (0.02-0.06 g/l);
CaCO₃ (0.5-1.0 g/l);
Red chlorophenol (0.04-0.1 g/l);
Skim milk (50-100 g/l);
NaCl (0.01-0.1 g/l);
Ammonium citric acid (0.5-2.0 g/l);
Ammonium acetic acid (1.0-3.0 g/l);
Ascorbic acid (0.1-0.5 g/l);
Sodium acetic acid (1.0-5.0 g/l);
MgSO₄·7H₂O (0.1-0.5 g/l);
MnSO₄·5H₂O (0.03-0.05 g/l);
Na₂HPO₄ (1.0-2.0 g/l);
KH₂PO₄ (0.3-0.7 g/l);
K₂HPO₄ (0.3-0.7 g/l);
FeSO₄·7H₂O (0.035-0.075 g/l);
Agar (0.1-15.0 g/l);
Micronutrients (0.5-1.0).

22. The method of claim 20, wherein a single strain is used or a biomass of strains from 2 to 100 human symbiont species are mixed, in equal proportions, or with a variation of up to 99.9% of a proportion.

23. The method of claim 22, wherein each strain is grown separately using a method generally accepted in biotechnology under conditions optimal for the species of microorganisms until the number of live cells is at least 1·10⁹ CFU/ml.

24. The method of claim 22, wherein the mixture of biomass of strains is sustained for at least 60 minutes at +20 to +28° C. to produce the metabolites before starting the release of the metabolites.

25. The method of claim 20, wherein the stress is the shock-cavitation wave.

26. The method of claim 20, wherein the stress is applied for 1 to 20 minutes.

27. The method of claim 20, wherein the separating includes producing a liquid sediment in multiple steps of centrifugal separation, while centrifugal acceleration value is varied from 3000 G to 21000 G, with a centrifugal exposure time from 1 min to 60 min and a temperature from +2 to +25° C.

28. The method of claim 27, wherein, after the separating, liquids are combined in proportions according to a material balance formula for mixing liquids of different densities:
$${V}_{k} \cdot {p}_{k} = {V}_{1} \cdot {p}_{1} + {V}_{2} \cdot {p}_{2} + {V}_{3} \cdot {p}_{3} + {V}_{n} \cdot {p}_{n} ,$$
where, V₁ and Vₙ are volumes of supernatant liquid No. 1 and n, in ml;
p₁ and pₙ are density of supernatant liquid No. 1 and n, g/ml; and
Vₖ is the final volume, ml; pk is the final density, g/ml.

29. The method of claim 20, further comprising mixing the released metabolites with an additive matrix.

30. The method of claim 20, further comprising sterilizing filtration after the separating.

31. The method of claim 20, further comprising adding an isotonic solution, and/or natural mineral water, and/or an aqueous solution of sea salt to the released protective metabolites.

32. The method of claim 20, further comprising impregnation of the anti-infective agent into wipes or patches, followed by a subsequent drying stage.

33. The method of claim 20, further comprising impregnation of the released metabolites into tampons, followed by a subsequent drying stage.

34. The method of claim 20, further comprising adding the released metabolites into a lozenge.

35. The method of claim 20, further comprising adding a suppository base to the released metabolites.

36. The method of claim 20, wherein the anti-infective agent is applicable to skin, and/or in fauces, and/or epipharynx, and/or external genital organs.

37. The method of claim 20, wherein the anti-infective agent is injectable into internal genital organs, and/or injectable into anus.
